(19) United States
(12) Patent Application Publication
(10) Pub. No.: US 2008/0279912 A1
(43) Pub. Date: Nov. 13, 2008

(54) Use of Cis-Epoxyeicosatrienoic Acids And Inhibitors of Soluble Epoxide Hydrolase to Alleviate Eye Disorders

(75) Inventors: Bruce D. Hammock, Davis, CA (US); Takaho Watanabe, Hadano (JP); Shirley J. Gee, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California Office of Technology Transfer, Oakland, CA (US)

(21) Appl. No.: 11/995,379

(22) PCT Filed: Jul. 12, 2006
(86) PCT No.: PCT/US06/27082

§ 371 Date: Jun. 9, 2008

Related U.S. Application Data
(60) Provisional application No. 60/698,661, filed on Jul. 12, 2005.

(51) Int. Cl. A61F 2/00; A61K 31/336; A61K 31/445; A61P 27/00; A61P 27/06; A61K 31/7105

(52) U.S. Cl. 424/427; 514/475; 514/315; 514/44

(57) ABSTRACT

The invention provides methods for alleviating eye disorders due to increased intraocular pressure (“IOP”) or inflammation by administering to the eye or eyes of an individual in need thereof a cis-epoxyeicosatrienoic acid, an inhibitor of soluble epoxide hydrolase (sEH), or both. The invention further provides for reducing IOP or inflammation by methods in which the sEH inhibitor or EETs, or both, are administered systemically. In some embodiments, the methods comprise administering to the individual a nucleic acid encoding an inhibitor of sEH.

### CROSS-REFERENCES TO RELATED APPLICATIONS

This disclosure claims priority from and the benefit of U.S. Provisional Application No. 60/698,661, filed Jul. 12, 2005, the contents of which are hereby incorporated by reference.

### STATEMENT AS TO RIGHTS TO INVENTIONS MADE UNDER FEDERALLY SPONSORED RESEARCH AND DEVELOPMENT

This invention was made with government support under Grant No. ES02710, awarded by the National Institutes of Health. The government has certain rights in the invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

A number of disorders affect the eye. For example, according to the National Eye Institute of the National Institutes of Health, nearly three million Americans have glaucoma, any of a group of eye diseases which cause optic atrophy and loss of visual field. Glaucoma is the second leading cause of blindness, and is more prevalent among persons over 60, African Americans over 40, and persons with a family history of glaucoma.

The most common glaucomas result from defects in drainage of fluids inside the eye. The front of the eye has a space called the anterior chamber. A clear fluid, known as the aqueous humor, flows in and out of the chamber, nourishing nearby tissues and draining debris. The fluid leaves the chamber at the “open angle,” where the cornea and iris meet. When the fluid reaches the angle, it flows through a spongy material, known as the trabecular meshwork, and leaves the eye through a channel called Schlemm's channel. If the drainage of the aqueous humor becomes blocked in the meshwork or Schlemm's channel, or if the aqueous humor is produced faster than it can be drained, the intraocular pressure (“IOP”) can increase, placing pressure on the retina and causing damage to the optic nerve.

“Open angle glaucoma” typically involves a drainage problem at the open angle, and is the most common form of glaucoma in the United States. While it is clear that IOP can damage the optic nerve, differences in physiology between individuals cause considerable variation in the pressure required before the nerve is damaged, with some individuals able to withstand for some time pressures that would blind another. In either case, however, it is desirable to reduce IOP to normal levels to prevent or to reduce damage to the nerve.

The second most common form is “closed angle glaucoma” (also called “angle closure glaucoma”), which can result from a hereditary defect or from an enlargement of the lens as the eye ages over the years. In closed angle glaucoma, the junction of cornea and iris is blocked or narrowed, resulting in increased IOP. While it is only one-fourth as common as open angle glaucoma, it is more prevalent in some populations, including the Japanese, Southeast Asians and Eskimos. Unlike open angle glaucoma, which occurs equally among men and women, closed angle glaucoma is far more prevalent in women than in men except among African Americans.

There is also a form of glaucoma, called “low-tension” or “normal tension glaucoma”, in which IOP is within normal limits. The cause of low-tension glaucoma is not clear, and has been attributed to such factors as unusual sensitivity of the optic nerve and to reduced blood supply to the nerve. Despite the fact that the IOP in this form of glaucoma is already considered to be within normal limits, it is considered desirable to reduce it further.

Among other disorders affecting the eye are dry eye syndrome, which is the most common complaint patients present to eye practitioners, and age-related macular degeneration, or “AMD.” As the name implies, AMD relates to the degeneration of the macula, the central portion of the retina packed with rod and cone cells necessary for fine vision. AMD is the major cause of severe vision loss among persons over 60 and occurs in two forms, the “dry” form and the “wet” form. Most AMD starts out as the dry form, in which a thinning of the retinal pigment epithelium (“RPE”), which is critical to delivering nutrients to and clearing wastes from the rods and cones. In particular, outer segments shed from the rods and cones and materials normally cleared by the RPE start to build up, causing deterioration of the cells. Dry AMD can progress to the wet form, which affects only 15% of persons with AMD, but it is responsible for the majority of the most serious vision loss. In the more common form of wet AMD, the choroid, the layer of blood vessels below the retina, forms new blood vessels (the formation of which is known as choroidal neovascularization, or “CNV”), which leak blood or plasma, thereby causing the vision to blur. CNV results in part in the formation of subretinal membrane formation. Another form of wet AMD, retinal pigment epithelial detachment (“PED”), is characterized not by the formation of new blood vessels but by leaking from the choroid, causing fluid buildup under the macula.

It would be useful to have additional methods of treating these and other eye disorders and conditions. The present invention fills these and other needs.

### BRIEF SUMMARY OF THE INVENTION

The invention relates to the discovery that inhibition of the enzyme soluble epoxide hydrolase is useful for reducing intraocular pressure (“IOP”), alleviating dry eye syndrome, and reducing progression of age-related macular degeneration (“AMD”). In a first group of embodiments, the invention provides methods of reducing intraocular pressure in an eye of an individual in need thereof, the method comprising administering to the individual by administration to the eye of an effective amount of an agent or agents selected from the group consisting of a cis-epoxyeicosatrienoic acid (“EET”), an inhibitor of soluble epoxide hydrolase (“sEH”), and a combination of an EET and an inhibitor of sEH, thereby reducing intraocular pressure. In some embodiments, the administration is by topical application of a liquid comprising the agent or agents. In some embodiments, the topical application is by instilling of the liquid into the conjunctival sac of the eye. In some embodiments, the administration is by topical administration of an ointment comprising the agent or agents. In some embodiments, the administration is by injection into the eye. In some embodiments, the individual has glaucoma. In some embodiments, the inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea. In some embodiments, the EET is selected from the group consisting of 14,15-EET, 8,9-EET and 11,12-EET. In some embodiments, the EET or the inhibitor of sEH, or both, are in a material which releases the EET, or inhibitor, or both, over time. In some embodiments, the inhibitor of sEH is a small interfering RNA which inhibits expression of sEH.

In another group of embodiments, the invention provides methods of alleviating “dry eye syndrome” in an individual in need thereof, the method comprising administration to an affected eye of said individual an effective amount of an agent or agents selected from the group consisting of a cis-epoxyeicosatrienoic acid (“EET”), an inhibitor of soluble epoxide hydrolase (“sEH”), and a combination of an EET and an inhibitor of sEH, thereby alleviating said dry eye syndrome. In some embodiments, the administration is by topical application of a liquid comprising the agent or agents. In some embodiments, the topical application is by instilling of the liquid into the conjunctival sac of the eye. In some embodiments, the administration is by topical administration of an ointment comprising the agent or agents. In some embodiments, the administration is by injection into the eye. In some embodiments, the inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea. In some embodiments, the EET is selected from the group consisting of 14,15-EET, 8,9-EET and 11,12-EET. In some embodiments, the EET or the inhibitor of sEH, or both, are in a material which releases the EET, or inhibitor, or both, over time. In some embodiments, the inhibitor of sEH is a small interfering RNA which inhibits expression of sEH.

In yet another group of embodiments, the invention provides methods of reducing progression of age-related macular degeneration (“AMD”) in an eye of an individual in need thereof, said method comprising administering to said individual by administration to said eye an effective amount of an agent or agents selected from the group consisting of a cis-epoxyeicosatrienoic acid (“EET”), an inhibitor of soluble epoxide hydrolase (“sEH”), and a combination of an EET and an inhibitor of sEH, thereby reducing progression of AMD in said eye. In some embodiments, the administration is by topical application of a liquid comprising said agent or agents. In some embodiments, the topical application is by instilling of the liquid into the conjunctival sac of the eye. In some embodiments, the administration is by topical administration of an ointment comprising said agent or agents. In some embodiments, the administration is by injection into the eye. In some embodiments, the inhibitor of sEH is a small interfering RNA which inhibits expression of sEH. In some embodiments, the inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea. In some embodiments, the EET is selected from the group consisting of 14,15-EET, 8,9-EET and 11,12-EET. In some embodiments, the EET or the inhibitor of sEH, or both, are in a material which releases the EET, or inhibitor, or both, over time. In some embodiments, the individual does not have an inflammatory disorder other than AMD or has been treated for an inflammatory disorder other than AMD with an agent which is not an EET or an inhibitor of sEH.

In still another group of embodiments, the invention provides methods of reducing intraocular pressure, alleviating dry eye syndrome, or of reducing progression of age-related macular degeneration (“AMD”), in an eye of an individual in need thereof. The methods comprise systemic administration to said individual of an effective amount of an agent or agents selected from the group consisting of an inhibitor of soluble epoxide hydrolase (“sEH”), and a combination of a cis-epoxyeicosatrienoic acid (“EET”) and an inhibitor of sEH, thereby reducing intraocular pressure, alleviating dry eye syndrome, or reducing progression of AMD. In some embodiments, the inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea. In some embodiments, the EET is selected from the group consisting of 14,15-EET, 8,9-EET and 11,12-EET. In some embodiments, the EET or the inhibitor of sEH, or both, are in a material which releases the EET, or inhibitor, or both, over time. In some embodiments, the individual has glaucoma. In some embodiments, the individual has dry eye syndrome. In some embodiments, the individual has AMD. In some embodiments, the AMD is wet AMD. In some embodiments, the inhibitor of sEH is an isolated nucleic acid which inhibits expression of a gene encoding soluble epoxide hydrolase (“sEH”). In some embodiments, the isolated nucleic acid is an small interfering RNA.

### BRIEF DESCRIPTION OF THE DRAWINGS

FIG. 1. Topical application of EETs and AUDA. Control IOP measurements were taken. A solution of 0.05 mL of 100 μg/mL EETs (diamonds) or 0.05 mL of 100 μg/mL AUDA (squares) was administered. Arrows indicate time of treatment (12 min for EETs and 50 min for AUDA).

### DETAILED DESCRIPTION OF THE INVENTION

### Introduction

A. Methods of using EETs and sEHI for treating eye disorders, including reducing intraocular pressure, alleviating dry eye syndrome, or slowing the progression of AMD. Surprisingly, it has now been discovered that intraocular pressure (“IOP”) can be reduced by administration to the eye of inhibitors of the enzyme known as soluble epoxide hydrolase (“sEH,” inhibitors of this enzyme are sometimes referred to herein as “sEHI”). Moreover, the effects of sEH inhibitors can be increased by also administering cis-epoxyeicosatrienoic acids (“EETs”). The effect is at least additive over administering the two agents separately, and may indeed be synergistic. Even more surprisingly, intraocular pressure can be reduced by administering EETs without also administering sEHIs.

In the studies underlying the present invention, rabbits whose eyes were treated topically or by injection into the eye with sEHI or EETs showed reduced intraocular pressure. It has previously been reported that sEH inhibitors can be used systemically to treat hypertension, and hypertension is one cause of increased IOP. The amount of the agent or agents applied to the eye in the present studies, however, would not be expected to reduce the animals' blood pressure even if disseminated into the systemic circulation. It is therefore believed that the effects observed are independent of any systemic anti-hypertensive effect of the sEHI or EETs administered.

Anti-hypertensive agents are not typically applied topically to the eyes and, if applied topically to any part of the body, would be applied in a transdermal carrier formulation intended to carry the agents through the skin into the systemic circulation. Further, it is likely that systemic levels of anti-hypertensive agents would not create high enough local concentrations in the eye to be therapeutically useful. In contrast, the methods of the present invention are intended to result in locally high concentrations of sEHI or EETs or both in the eye, even though there may be some incidental diffusion or transport of the agents into the systemic circulation. It is therefore neither expected nor intended that the methods of the present invention will result in levels of the agents in the systemic circulation that would be considered to be effective in treating hypertension.

Further, the topical administration of sEHI or of EETs, or of both, of the methods of the present invention can, for example, be used to reduce intraocular pressure in persons who have normal or low blood pressure and who would therefore not be treated for hypertension. Indeed, the rabbits in the studies reported in the Examples were not hypertensive. Thus, the reductions in IOP observed after application of the sEHI or EETs in these studies could not be due to a reduction of hypertension in these animals.

Based on the results obtained with reducing IOP, it is believed that the methods of the invention can also be used to treat “dry eye syndrome,” one of the most common complaints seen by eye practitioners. According to the National Eye Institute (“NEI”), dry eye syndrome is usually characterized by a scratchy or sandy feeling as if something is in the eye. The NEI indicates that other symptoms may include stinging or burning of the eye; episodes of excess tearing that follow periods of very dry sensation; a stringy discharge from the eye; and pain and redness of the eye. Persons with the syndrome may also experience heaviness of the eyelids or blurred, changing, or decreased vision, although loss of vision is uncommon.

These problems can arise because the patient is producing fewer tears, a common effect of aging, or because the lipid and mucin layers produced by the eye are of such poor quality that tears cannot remain in the eye long enough to keep the eye sufficiently lubricated. Dry eye syndrome from these causes is typically treated by the use of artificial tears and by plugging the tear ducts to prevent the tear layer from draining, but may also benefit from the application of sEHI or EETs, or combinations of the two. In some cases, however, dry eye syndrome can be caused by an inflammation, either of the lacrimal gland or of the surface of the eye. This condition is treated with anti-inflammatory agents. Given the anti-inflammatory properties of sEHI when administered systemically, it is expected that sEHI and EETs, or combinations of the two, will be especially useful in treating dry eye syndrome resulting from inflammation.

It is further expected that sEHI and EETs, or combinations of the two, will be useful in treating age-related macular degeneration (“AMD”). Most, if not all, treatments for AMD are for the wet form, and include photocoagulation, in which a high energy laser is used to seal off new blood vessels in the choroid and photodynamic therapy, in which a cold laser is used to activate a photosensitive drug that preferentially binds to new vasculature, closing the new blood vessels. Two newer therapies inhibit the action of vascular endothelial growth factor (“VEGF”), thereby decreasing the formation and growth of new blood vessels. Macugen®, a pegylated anti-VEGF aptamer, is now approved by the FDA for sale in the United States. Lucentis®, an anti-VEGF antibody, is also now approved for sale by the FDA. Both agents are administered by intravitreal injection (that is, by injection into the vitreous humor, the gel of water and collagen in the cavity of the eye between the lens and the retina). Yet another drug, Sirna-027 (Sirna Therapeutics, Inc., San Francisco, Calif.), a chemically modified short interfering RNA (“siRNA”) inhibitor of VEGF administered by intravitreal injection, was reported to show significant activity in retarding wet AMD in a Phase I clinical trial in 25 patients. The methods of the present invention can be used alone or in combination with one or more of the therapies described above.

Recent studies suggest that there may be a tie between inflammatory responses and AMD. The levels of the inflammatory marker C-reactive protein (“CRP”) were found to be significantly higher in persons with advanced AMD than in those without AMD, even after taking into account variables such as age, sex, smoking and body mass index, while increased levels of CRP were associated with increased risk for intermediate and advanced AMD. levels. (Seddon et al., Am J Opthamol 138(4):704-705 (2004)). In another study, an antibody to tumor necrosis factor (“TNF”) was found to cause regression of subretinal membrane formed by choroidal neovascularization (“CNV”). (Markomichelakis et al., Am J Opthalmol 139(3):537-540 (2005)). The patients in the study had no evidence of past or present intraocular inflammation. While the authors believe this effect is likely due to anti-angiogenic properties of the drug, we believe that TNF is a pro-inflammatory marker and that at least some of the effect of the anti-TNF antibody seen in the study was due to anti-inflammatory action despite the fact that no intraocular inflammation could be observed visually.

Work from the laboratory of the present inventors has demonstrated that sEHI and EETs have anti-inflammatory properties. We expect that sEHIs and EETs, alone or in combination, will slow or reverse the progression of wet AMD and may reduce the progression of dry AMD to wet AMD. It is believed that topical application of sEHIs, EETs, or combinations of sEHI and EETs will be useful for these purposes, since it is expected that the agent or agents will diffuse through the aqueous humor and vitreous humor to the retinal surface. In some preferred embodiments, the sEHIs, EETs, or combinations of sEHI and EETs are introduced into the subject eye by intravitreal injection, as are the anti-AMD agents noted above, to permit a higher concentration of the agents to reach the surface of the retina and, in particular, of the macula. Lack of progression of wet AMD can be assessed clinically by standard techniques, such as by periodically repeating fluorangiographic assessments of CNV and by observing the extent of subretinal membranes; an increase in the membranes indicates progression of the AMD, while regression indicates lack of progression or actual improvement.

It is also worth noting that diabetic retinopathy, age-related macular degeneration and corneal graft rejection are characterized by vascular leakage and angiogenesis. The earliest event associated with the progression of diabetic retinopathy is ischemia in the retinal blood vessels resulting in a local inflammatory response. Upregulation of COX-2 is known in diabetic subjects with proliferative retinopathy. Elevated COX-2 and constitutively expressed COX-1 metabolize arachidonic acid to prostaglandins that induce the expression of vascular endothelial growth factor (VEGF) which is responsible for the initiation and progression of diabetic retinopathy. Because this pathway is similar to treatments for age-related macular degeneration, it is believed that treatment with EETs, sEHI, or a combination thereof can be used alone or in conjunction with treatments that reduce VEGF in diabetic retinopathy, corneal graft rejections and other eye diseases that are a result of neovascularization.

Persons Who can Benefit from Use of EETs or sEHI or Both

The methods of the present invention relate to the application of EETs, sEHI, or combinations thereof, in eye drops, ointments, intraocular injections, or other formulations intended for application to or into the eyes. While systemic use of anti-hypertensive agents may indirectly reduce IOP, it does not appear that anti-hypertensive agents are applied topically to the eye to reduce IOP. Similarly, while work from our laboratory has previously shown that EETs and sEHI have anti-inflammatory properties, that work did not show that topical application to the eyes would have any effect, nor that high local concentrations in the eye would treat eye conditions, whether due to inflammation or other sources.

It is not expected that systemic administration of EETs by themselves will result in local concentrations of the EETs in the eye to be high enough to reduce IOP, to reduce dry eye syndrome, or to slow or reverse progression of wet AMD. Thus, if it is desired to reduce IOP, alleviate dry eye syndrome, or slow the progression of AMD, by systemic administration of EETs, the EETs should be administered in combination with an sEHI or as a stabilized analog. If the sEHI has a range of doses for systemic administration, it should be administered in doses at the higher end of the range to afford as high as possible a concentration in the eye.

Hypertension can, of course, be treated with agents other than EETs and sEHI. The present invention, however, shows that treatment of hypertension with sEHI, EETs, or both, is likely to have a direct effect in reducing intraocular pressure in the patient, and is therefore to be preferred over the use of other anti-hypertensive agents. Similarly, while inflammation can be treated with agents other than EETs and sEHI, treatment of inflammation with sEHI, EETs, or both, is likely to have an effect in reducing the progression of AMD in patients over 60 or otherwise at risk for AMD, and is therefore to be preferred over the use of other anti-inflammatory agents. Similarly, the use of EETs, sEHI, or combinations thereof are to be preferred over other agents that can be used to reduce diabetic retinopathy.

In some preferred embodiments, however, the person being treated systemically with EETs, sEHI, or both, to reduce IOP, alleviate dry eye syndrome, or to slow or reverse progression of AMD does not have hypertension, if he or she has hypertension, has not been treated for this condition with an sEHI or EET. Further, in preferred embodiments, the patient does not have uveitis or is not being treated for this condition with an sEHI or EET. In some preferred embodiments, the person being treated to reduce IOP does not have an inflammation or, if he or she has an inflammation, has not been, or is not being treated, for this condition with an sEH inhibitor or EET. In some embodiments, the person has an inflammation but is being treated for that inflammation by an anti-inflammatory agent, such as a steroid, that is not an inhibitor of sEH. Whether or not any particular anti-inflammatory agent or anti-hypertensive agent is also an sEH inhibitor can be readily determined by standard assays, such as those taught in U.S. Pat. No. 5,955,496.

In some preferred embodiments, the patient to be treated to reduce IOP, to slow progression of AMD or to relieve dry eye syndrome, does not also have a disease or condition caused by an autoimmune disease or a disorder associated with a T-lymphocyte mediated immune function autoimmune response. In some embodiments, the patient does not also have a pathological condition selected from type 1 or type 2 diabetes, insulin resistance syndrome, atherosclerosis, coronary artery disease, angina, ischemia, ischemic stroke, Raynaud's disease, or renal disease. In some embodiments, the patient is not a person with diabetes mellitus whose blood pressure is 130/80 or less, a person with metabolic syndrome whose blood pressure is less than 130/85, a person with a triglyceride level over 215 mg/dL, or a person with a cholesterol level over 200 mg/dL or is a person with one or more of these conditions who is not taking an inhibitor of sEH. In some embodiments, the patient does not have an obstructive pulmonary disease, an interstitial lung disease, or asthma. In some embodiments, the patient is not also being treated with an inhibitor of one or more enzymes selected from the group consisting of cyclo-oxygenase (“COX”)-1, COX-2, and 5-lipoxygenase (“5-LOX”).

Medicaments of EETs can be made which can be administered by themselves or in conjunction with one or more sEH inhibitors, or a medicament containing one or more sEH inhibitors can optionally contain one or more EETs. The EETs can be administered alone, or concurrently with a sEH inhibitor or following administration of a sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs administered after an sEH inhibitor are intended to be administered while the sEH inhibition is still in effect, therefore, it is desirable that the EETs be administered during the period during which the inhibitor will be present in amounts to be effective to delay hydrolysis of the EETs. Typically, in such a situation, the EET or EETs will be administered within 48 hours of administering an sEH inhibitor. More preferably, where the effect of the EET or EETs is intended to be enhanced by the effect of an sEHI, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In some embodiments, the sEH inhibitor may be a nucleic acid, such as a small interfering RNA (siRNA) or a micro RNA (miRNA), which reduces expression of a gene encoding sEH. As noted above, an siRNA directed to VEGF has shown efficacy against wet AMD in a Phase I clinical trial. The EETs may be administered in combination with such a nucleic acid. Typically, a study will determine the time following administration of the nucleic acid before a decrease is seen in levels of sEH. The EET or EETs will typically then be administered a time calculated to be after the activity of the nucleic acid has resulted in a decrease in sEH levels.

In some embodiments, the EETs, the sEH inhibitor, or both, are provided in a material that permits them to be released over time to provide a longer duration of action. Slow release coatings are well known in the pharmaceutical art; the choice of the particular slow release coating is not critical to the practice of the present invention.

### DEFINITIONS

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“cis-Epoxyeicosatrienoic acids” (“EETs”) are biomediators synthesized by cytochrome P450 epoxygenases. As discussed further below, while in some embodiments the use of unmodified EETs is preferred, derivatives of EETs, such as amides and esters, EETs analogs, EETs mimics, and EETs optical isomers can all be used in the methods of the invention, either by themselves (e.g., just an EET analog) or as mixtures of two or more of these forms (e.g., an unmodified EET and an EET analog). For convenience of reference, the term “EETs” as used herein refers to all of these forms unless otherwise required by context.

“Epoxide hydrolases” (“EHs;” EC 3.3.2.3) are enzymes in the alpha beta hydrolase fold family that add water to 3 membered cyclic ethers termed epoxides. The addition of water to the epoxides results in the corresponding 1,2-diols (Hammock, B. D. et al., in Comprehensive Toxicology Biotransformation (Elsevier, N.Y.), pp. 283-305 (1997); Oesch, F. Xenobiotica 3:305-340 (1972)). Four principal EH's are known: leukotriene epoxide hydrolase, cholesterol epoxide hydrolase, microsomal EH (“mEH”), and soluble EH (“sEH,” previously called cytosolic EH). The leukotriene EH acts on leukotriene A4, whereas the cholesterol EH hydrates compounds related to the 5,6-epoxide of cholesterol. The microsomal epoxide hydrolase metabolizes monosubstituted, 1,1-disubstituted, cis-1,2-disubstituted epoxides and epoxides on cyclic systems to their corresponding diols. Because of its broad substrate specificity, this enzyme is thought to play a significant role in ameliorating epoxide toxicity. Reactions of detoxification typically decrease the hydrophobicity of a compound, resulting in a more polar and thereby excretable substance.

“Soluble epoxide hydrolase” (“sEH”) is an epoxide hydrolase which in many cell types converts EETs to dihydroxy derivatives called dihydroxyeicosatrienoic acids (“DHETs”). The cloning and sequence of the murine sEH is set forth in Grant et al., J. Biol. Chem. 268(23): 17628-17633 (1993). The cloning, sequence, and accession numbers of the human sEH sequence are set forth in Beetham et al., Arch. Biochem. Biophys. 305(1):197-201 (1993). The amino acid sequence of human sEH is found in U.S. Pat. No. 5,445,956 and is set forth as SEQ ID NO.: 1 herein; the nucleic acid sequence encoding the human sEH is also set forth in that patent and is SEQ ID NO.:2 herein. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Soluble EH is only very distantly related to niEH and hydrates a wide range of epoxides not on cyclic systems. In contrast to the role played in the degradation of potential toxic epoxides by mEH, sEH is believed to play a role in the formation or degradation of endogenous chemical mediators. Unless otherwise specified, as used herein, the terms “soluble epoxide hydrolase” and “sEH” refer to human sEH.

Unless otherwise specified, the term “sEH inhibitor” (also abbreviated as “sEHI”) as used herein refers to an inhibitor of human sEH. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester or salt of that compound) that retain activity as an sEH inhibitor.

“Topical application” to the eye refers to the administration of an agent to the eye by applying the agent to the eyelids or to the conjunctival sac in aqueous or viscous solutions or suspensions, in ointments, as fine powders, on cotton pledgets, by drug-impregnated contact lenses, by injection into the eye, by mechanical pumps, or by membrane release systems.

“Dry eye syndrome” is well known in the art and is one of the most common complaints patients present to eye professionals. The condition is usually diagnosed on the basis of history, but can be confirmed through physical examination, which will reveal a reduced tear volume and short tear “break up” time. Typically, dyes are used to visualize the tear layer to facilitate examination and diagnosis.

By “physiological conditions” is meant an extracellular milieu having conditions (e.g., temperature, pH, and osmolarity) which allows for the sustenance or growth of a cell of interest.

“Micro-RNA” (“miRNA”) refers to small, noncoding RNAs of 18-25 nt in length that negatively regulate their complementary mRNAs at the posttranscriptional level in many eukaryotic organisms. See, e.g., Kurihara and Watanabe, Proc Natl Acad Sci USA 101(34):12753-12758 (2004). Micro-RNA's were first discovered in the roundworm C. elegans in the early 1990s and are now known in many species, including humans. As used herein, it refers to exogenously administered miRNA unless specifically noted or otherwise required by context.

### Intraocular Pressure

The eye does not collapse because the pressure within the eye (the “intraocular pressure,” or “IOP”) is greater than that of the surrounding atmosphere. Normally, the IOP is between 10-20 mm Hg greater than the pressure of the atmosphere, although there is some modest daily fluctuation. IOP is created by the aqueous humor, a clear fluid that enters the anterior chamber of the eye via the ciliary body epithelium (inflow), flows through the anterior segment bathing the lens, iris, and cornea, and then leaves the eye via specialized tissues known as the trabecular meshwork and Schlemm's canal to flow into the venous system. Intraocular pressure is maintained by a balance between fluid secretion and fluid outflow. According to the NEI, most glaucomas result from a defect in the outflow and a subsequent buildup of pressure.

IOP is usually measured by determining the resistance of the eye to an external force. A variety of instruments are used to measure IOP clinically, including the Goldmann tonometer, which uses a prism to flatten the cornea, the Tono-Pen® XL applanation tonometer (Medtronic Xomed Ophthalmics, Inc., Jacksonville, Fla.), a hand-held device containing a plunger, and the Schiotz tonometer, which measures the indentation of the cornea produced by a weight.

### Inhibitors of Soluble Epoxide Hydrolase

Scores of sEH inhibitors are known, of a variety of chemical structures. Derivatives in which the urea, carbamate, or related amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U. S. A, 96:8849-8854 (1999), which provides substantial guidance on designing urea derivatives that inhibit the enzyme.

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-adamantyl ureas have been tested and have about the same high activity as an inhibitor of sEH.) Thus, isomers of adamantyl dodecyl urea are preferred inhibitors. It is further expected that N,N′-dodecyl-cyclohexyl urea (DCU), and other inhibitors of sEH, and particularly dodecanoic acid ester derivatives of urea, are suitable for use in the methods of the invention. Preferred inhibitors include:
12-(3-Adamantan-1-yl-ureido)dodecanoic acid (AUDA)

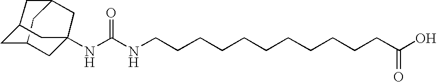

12-(3-Adamantan-1-yl-ureido)dodecanoic acid butyl ester (AUDA-BE)

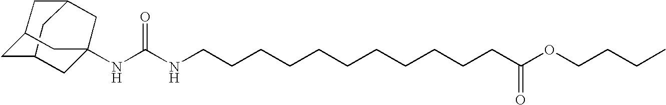

### (For Use in Aqueous Media, the Free Acid Form of AUDA is Preferred to the Butyl Ester.)

Adamantan-1-yl-3-{5-[2-(2-ethoxyethoxy)ethoxy]pentyl}urea (compound 950)

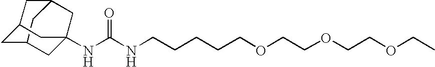

Another preferred group of inhibitors are piperidines. The following Table sets forth some exemplar piperidines and their ability to inhibit sEH activity, expressed as the amount needed to reduce the activity of the enzyme by 50% (expressed as “IC₅₀”).

TABLE 1
IC₅₀ values for selected alkylpiperidine-based sEH inhibitors

| | | n = 0 | | n = 1 | |
|---|---|---|---|---|---|
| 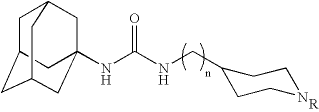 | | Compound | IC₅₀ (μM)^{a} | Compound | IC₅₀ (μM)^{a} |
| R: | H | I | 0.30 | II | 4.2 |
| | 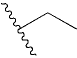 | 3a | 3.8 | 4.a | 3.9 |
| | 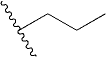 | 3b | 0.81 | 4b | 2.6 |
| | 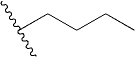 | 3c | 1.2 | 4c | 0.61 |
| | 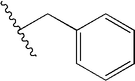 | 3d | 0.01 | 4d | 0.11 |

^{a}As determined via a kinetic fluorescent assay.

A number of other inhibitors, each of which is preferred for use in the methods and compositions of the invention, are set forth in co-owned applications PCT/US2004/010298 and U.S. Published Patent Application Publication 2005/0026844. For those methods in which ocular application is used, it is preferable if the inhibitor chosen has or is modified to have increased water solubility and other parameters to facilitate solubility in artificial tears and penetration of membranes.

U.S. Pat. No. 5,955,496 (the '496 patent) sets forth a number of epoxide hydrolase inhibitors suitable for use in the methods of the invention. One category of inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S, S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods of the invention are set forth in U.S. Pat. Nos. 6,150,415 (the '415 patent) and 6,531,506 (the '506 patent). Two preferred classes of inhibitors of the invention are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty inhibitors of Formula 2, which were shown to inhibit human sEH at concentrations as low as 0.1 μM. Any particular inhibitor can readily be tested to determine whether it will work in the methods of the invention by standard assays, such as that set forth in the Examples, below. Esters and salts of the various compounds discussed above or in the cited patents, for example, can be readily tested by these assays for their use in the methods of the invention.

As noted above, chalcone oxides can serve as an alternate substrate for the enzyme. While chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., MEDICINAL CHEMISTRY: AN INTRODUCTION, John Wiley & Sons Ltd. (West Sussex, England, 2000)). Since the uses of the invention contemplate inhibition of sEH over periods of time which can be measured in days, weeks, or months, chalcone oxides, and other inhibitors which have a half life whose duration is shorter than the practitioner deems desirable, are preferably administered in a manner which provides the agent over a period of time. For example, the inhibitor can be provided in materials that release the inhibitor slowly, to provide a high local concentration in or near the eye. Methods of administration that permit high local concentrations of an inhibitor over a period of time are known, and are not limited to use with inhibitors which have short half lives although, for inhibitors with a relatively short half life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., THE PHARMACOLOGICAL BASIS OF THERAPEUTICS, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, the sEH inhibitor used in the methods taught herein is a “soft drug.” Soft drugs are compounds of biological activity that are rapidly inactivated by enzymes as they move from a chosen target site. EETs and simple biodegradable derivatives administered to the eye may be considered soft drugs in that they are likely to be enzymatically degraded by sEH as they diffuse away from the eye following ocular administration. Some sEHI, however, may diffuse or be transported following ocular administration to regions where their activity in inhibiting sEH may not be desired. Thus, multiple soft drugs for ocular treatment have been prepared. These include but are not limited to carbamates, esters, carbonates and amides placed in the sEHI, approximately 7.5 angstroms from the carbonyl of the central pharmacophore. These are highly active sEHI that yield biologically inactive metabolites by the action of esterase and/or amidase. Groups such as amides and carbamates on the central pharmacophores also increase solubility in artificial tears and generate soft drugs. Similarly, easily metabolized ethers may contribute soft drug properties and also increase the solubility.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

Preferably, the inhibitor inhibits sEH without also significantly inhibiting microsomal epoxide hydrolase (“mEH”). Preferably, at concentrations of 500 μM, the inhibitor inhibits sEH activity by at least 50% while not inhibiting mEH activity by more than 10%. Preferred compounds have an IC₅₀ (inhibition potency or, by definition, the concentration of inhibitor which reduces enzyme activity by 50%) of less than about 500 μM. Inhibitors with lower IC₅₀s are more preferred than are inhibitors having higher IC₅₀s, so that inhibitory concentrations can be achieved when the inhibitor is administered therapeutically to a subject, such as a person in need thereof. Thus, inhibitors with IC₅₀s of less than 400 μM are preferred, inhibitors with IC₅₀s of less than 300 μM are more preferred, inhibitors with IC₅₀s of less than 200 μM are still more preferred, while inhibitors with IC₅₀s of less than 100 μM are even more preferred. Inhibitors with IC₅₀s of less than 80 μM, 75 μM, 60 μM, 50 μM, 40 μM, 30 μM, 25 μM, 20 μM, 15 μM, 10 μM, 5 μM, 3 μM, 2 μM, 1 μM or even less are more preferred, with an inhibitors having a lower IC₅₀ being more preferred than an inhibitor with a higher IC₅₀. Assays for determining the IC₅₀ of any given sEH inhibitor are known in the art and described elsewhere herein.

### EETs

EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into dihydroxyeicosatrienoic acids (“DHETs”) is reduced.

It has long been believed that EETs administered systemically would be hydrolyzed too quickly by endogenous sEH to be helpful. In the only prior report of therapeutic administration of EETs of which we are aware, EETs were administered by catheters inserted into mouse aortas. The EETs were infused continuously during the course of the experiment because of concerns over the short half life of the EETs. See, Liao and Zeldin, International Publication WO 01/10438 (hereafter “Liao and Zeldin”). It also was not known whether endogenous sEH could be inhibited sufficiently in body tissues to permit administration of exogenous EET to result in increased levels of EETs over those normally present. Further, it was thought that EETs, as epoxides, would be too labile to survive the storage and handling necessary for therapeutic use.

In studies from the laboratory of one of the present inventors, however, it has been shown that systemic administration of EETs in conjunction with inhibitors of sEH had better results than did administration of sEH inhibitors alone. EETs were not administered by themselves in these studies since it was anticipated they would be degraded too quickly to have a useful effect. Additional studies from the laboratory of one of the present inventors have now shown, however, that administration of EETs by themselves has had therapeutic effect. Without wishing to be bound by theory, it is surmised that topical EETs avoid the first pass hepatic metabolism and the exogenous EETs overwhelm endogenous sEH, and allows EETs levels to be increased for a sufficient period of time to have therapeutic effect. Thus, EETs can be administered without also administering an sEHI to provide a therapeutic effect. Moreover, we have found that EETs, if not exposed to acidic conditions or to sEH are stable and can withstand reasonable storage, handling and administration. Thus, EETs administered topically to the eyes are expected to be stable for such use.

In short, sEHI, EETs, or co-administration of sEHIs and of EETs, can be used to inhibit the development of, or to reduce, intraocular pressure or dry eye syndrome or to slow the progression of age-related macular degeneration, diabetic retinopathy, corneal graft rejection, or other eye diseases that are a result of neovascularization. In some embodiments, one or more EETs are administered to the patient without also administering an sEHI. In some embodiments, one or more EETs are administered shortly before or concurrently with administration of an sEH inhibitor to slow hydrolysis of the EET or EETs. In some embodiments, one or more EETs are administered after administration of an sEH inhibitor, but before the level of the sEHI has diminished below a level effective to slow the hydrolysis of the EETs.

EETs useful in the methods of the present invention include 14,15-EET, 8,9-EET and 11,12-EET, and 5,6 EETs. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

If desired, EETs analogs, mimics, or derivatives that retain activity can be used in place of or in combination with unmodified EETs. Liao and Zeldin, supra, define EET analogs as compounds with structural substitutions or alterations in an EET, and include structural analogs in which one or more EET olefins are removed or replaced with acetylene or cyclopropane groups, analogs in which the epoxide moiety is replaced with in chain ethers, oxitane, or furan rings and heteroatom analogs. In other analogs, the epoxide moiety is replaced with ether, alkoxides, difluorocyclopropane, or carbonyl, while in others, the carboxylic acid moiety is replaced with a commonly used mimic, such as a nitrogen heterocycle, a sulfonamide, or another polar functionality. In preferred forms, the analogs or derivatives are relatively stable as compared to an unmodified EET because they are more resistant than an EET to sEH and to chemical breakdown. “Relatively stable” means the rate of hydrolysis by sEH is at least 25% less than the hydrolysis of the unmodified EET in a hydrolysis assay, more preferably 50% or more lower than the rate of hydrolysis of an unmodified EET. Liao and Zeldin show, for example, episulfide and sulfonamide EETs derivatives. Amide and ester derivatives of EETs and that are relatively stable are preferred embodiments. Mimics are compounds in which the epoxide of an EET is replaced with an ether, such as methoxide, ethoxide, or propoxide.

In preferred forms, the mimics, analogs or derivatives have the biological activity of the unmodified EET regioisomer from which they are modified or derived, in reducing intraocular pressure, relieving dry eye syndrome, slowing the progression of macular degeneration, slowing the progression of diabetic retinopathy, or slowing the rejection of a corneal graft. Whether or not a particular EET analog, mimic, or derivative has the biological activity of the unmodified EET can be readily determined by using it in the assays described in the Examples. As mentioned in the Definition section, above, for convenience of reference, the term “EETs” as used herein refers to unmodified EETs, and EETs analogs, mimics, and derivatives unless otherwise required by context.

In some embodiments, the EET or EETs are embedded or otherwise placed in a material that releases the EET over time. Materials suitable for promoting the slow release of compositions such as EETs are known in the art. Optionally, one or more sEH inhibitors may also be placed in the slow release material. The EET, EETs, sEHI or combination of EETs and sEHI can also be placed in the slow release material to treat dry eye symptoms. For example, the EETs, sEHI, or combinations thereof, can be embedded in or otherwise placed on or in small pellets of hydroxypropyl cellulose (more formally known as cellulose, 2-hydroxypropyl ether) to be placed under the eyelid. Pellets of hydroxypropyl cellulose are commercially available under the name LACRISERT® (Merck & Co., Inc., Whitehouse Station, N.J.) for ophthalmic use to alleviate dry eye symptoms. As another example, in some cases, the opening of the tear drain in the eyelid is temporarily stopped with a dissolvable collagen plug called a punctal plug. The EETs or sEHI or combinations thereof can be embedded in or otherwise placed on or in the plug to release over time.

For systemic administration, the EET or EETs can be administered orally. Since EETs are subject to degradation under acidic conditions, EETs intended for oral administration can be coated with a coating resistant to dissolving under acidic conditions, but which dissolve under the mildly basic conditions present in the intestines. Suitable coatings, commonly known as “enteric coatings” are widely used for products, such as aspirin, which cause gastric distress or which would undergo degradation upon exposure to gastric acid. By using coatings with an appropriate dissolution profile, the coated substance can be released in a chosen section of the intestinal tract. For example, a substance to be released in the colon is coated with a substance that dissolves at pH 6.5-7, while substances to be released in the duodenum can be coated with a coating that dissolves at pH values over 5.5. Such coatings are commercially available from, for example, Rohm Specialty Acrylics (Rohm America LLC, Piscataway, N.J.) under the trade name “Eudragit®”. The choice of the particular enteric coating is not critical to the practice of the invention.

### Assays for Epoxide Hydrolase Activity

Any of a number of standard assays for determining epoxide hydrolase activity can be used to determine inhibition of sEH. For example, suitable assays are described in Gill, et al., Anal Biochem 131, 273-282 (1983); and Borhan, et al., Analytical Biochemistry 231, 188-200 (1995)). Suitable in vitro assays are described in Zeldin et al., J. Biol. Chem. 268:6402-6407 (1993). Suitable in vivo assays are described in Zeldin et al., Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Fla., pp. 303-311 and Wixtrom et al., In: Biochemical Pharmacology and Toxicology, Vol. 1: Methodological Aspects of Drug Metabolizing Enzymes, (Zakim, D. and D. A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, supra, and Hammock. Anal. Biochem. 174:291-299 (1985) and Dietze, et al. Anal. Biochem. 216:176-187 (1994)).

The enzyme also can be detected based on the binding of specific ligands to the catalytic site which either immobilize the enzyme or label it with a probe such as dansyl, fluorescein, luciferase, green fluorescent protein or other reagent. The enzyme can be assayed by its hydration of EETs, its hydrolysis of an epoxide to give a colored product as described by Dietze et al., 1994, supra, or its hydrolysis of a radioactive surrogate substrate (Borhan et al., 1995, supra). The enzyme also can be detected based on the generation of fluorescent products following the hydrolysis of the epoxide. Numerous method of epoxide hydrolase detection have been described (see, e.g., Wixtrom, supra).

The assays are normally carried out with a recombinant enzyme following affinity purification. They can be carried out in crude tissue homogenates, cell culture or even in vivo, as known in the art and described in the references cited above.

### Other Means of Inhibiting sEH Activity

Other means of inhibiting sEH activity or gene expression can also be used in the methods of the invention. For example, a nucleic acid molecule complementary to at least a portion of the human sEH gene can be used to inhibit sEH gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science. 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet. 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically-inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo. In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; nucleotides 42-1703 of SEQ ID NO:1 are the nucleic acid sequence encoding the amino acid sequence.

A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research on the internet by entering “http://” followed by “jura.wi.mit.edu/pubint/http://iona.wi.mit.edu/siRNAext/.”

For example, using the program available from the Whitehead Institute, the following sEH target sequences and siRNA sequences can be generated:

| | |
|---|---|
| 1) Target: | |
| CAGTGTTCATTGGCCATGACTGG | (SEQ ID NO: 3) |
| Sense-siRNA: | |
| 5′-GUGUUCAUUGGCCAUGACUTT-3′ | (SEQ ID NO: 4) |
| Antisense-siRNA: | |
| 5′-AGUCAUGGCCAAUGAACACTT-3′ | (SEQ ID NO: 5) |
| 2) Target: | |
| GAAAGGCTATGGAGAGTCATCTG | (SEQ ID NO: 6) |
| Sense-siRNA: | |
| 5′-AAGGCUAUGGAGAGUCAUCTT-3′ | (SEQ ID NO: 7) |
| Antisense-siRNA: | |
| 5′-GAUGACUCUCCAUAGCCUUTT-3′ | (SEQ ID NO: 8) |
| 3) Target | |
| AAAGGCTATGGAGAGTCATCTGC | (SEQ ID NO: 9) |
| Sense-siRNA: | |
| 5′-AGGCUAUGGAGAGUCAUCUTT-3′ | (SEQ ID NO: 10) |
| Antisense-siRNA: | |
| 5′-AGAUGACUCUCCAUAGCCUTT-3′ | (SEQ ID NO: 11) |
| 4) Target: | |
| CAAGCAGTGTTCATTGGCCATGA | (SEQ ID NO: 12) |
| Sense-siRNA: | |
| 5′-AGCAGUGUUCAUUGGCCAUTT-3′ | (SEQ ID NO: 13 |
| Antisense-siRNA: | |
| 5′-AUGGCCAAUGAACACUGCUTT-3′ | (SEQ ID NO: 14 |
| 5) Target: | |
| CAGCACATGGAGGACTGGATTCC | (SEQ ID NO: 15) |
| Sense-siRNA: | |
| 5′-GCACAUGGAGGACUGGAUUTT-3′ | (SEQ ID NO: 16) |
| Antisense-siRNA: | |
| 5′-AAUCCAGUCCUCCAUGUGCTT-3′ | (SEQ ID NO: 17) |

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplar vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO: 18). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31 (15):e77 (2003).

As an example, the siRNA targets identified above can be targeted by hairpin siRNA as follows. To attack the same targets by short hairpin RNAs, produced by a vector (permanent RNAi effect), sense and antisense strand can be put in a row with a loop forming sequence in between and suitable sequences for an adequate expression vector to both ends of the sequence. The following are non-limiting examples of hairpin sequences that can be cloned into the pSuper vector:

| | |
|---|---|
| 1) Target: | |
| CAGTGTTCATTGGCCATGACTGG | (SEQ ID NO: 19) |
| Sense strand: | |
| 5′-GATCCCCGTGTTCATTGGCCATGACTTTCAA | (SEQ ID NO: 20) |
| GAGAAGTCATGGCCAATGAACACTTTTT-3′ | |
| Antisense strand: | |
| 5′-AGCTAAAAAGTGTTCATTGGCCATGACTTCT | (SEQ ID NO: 21) |
| CTTGAAAGTCATGGCCAATGAACACGGG-3′ | |
| 2) Target: | |
| GAAAGGCTATGGAGAGTCATCTG | (SEQ ID NO: 22) |
| Sense strand: | |
| 5′-GATCCCCAAGGCTATGGAGAGTCATCTTCAA | (SEQ ID NO: 23) |
| GAGAGATGACTCTCCATAGCCTTTTTTT-3′ | |
| Antisense strand: | |
| 5′-AGCTAAAAAAAGGCTATGGAGAGTCATCTCT | (SEQ ID NO: 24) |
| CTTGAAGATGACTCTCCATAGCCTTGGG-3′ | |
| 3) Target: | |
| AAAGGCTATGGAGAGTCATCTGC | (SEQ ID NO: 25) |
| Sense strand: | |
| 5′-GATCCCCAGGCTATGGAGAGTCATCTTTCAA | (SEQ ID NO: 26) |
| GAGAAGATGACTCTCCATAGCCTTTTTT-3′ | |
| Antisense strand: | |
| 5′- | |
| AGCTAAAAAAGGCTATGGAGAGTCATCATCTCTT | (SEQ ID NO: 27) |
| GAAAGATGACTCTCCATAGCCTGGG-3′ | |
| 4) Target: | |
| CAAGCAGTGTTCATTGGCCATGA | (SEQ ID NO: 28) |
| Sense strand: | |
| 5′-GATCCCCAGCAGTGTTCATTGGCCATTTCAA | (SEQ ID NO: 29) |
| GAGAATGGCCAATGAACACTGCTTTTTT-3′ | |
| Antisense strand: | |
| 5′-AGCTAAAAAAGCAGTGTTCATTGGCCATTCT | (SEQ ID NO: 30) |
| CTTGAAATGGCCAATGAACACTGCTGGG-3′ | |
| 5) Target: | |
| CAGCACATGGAGGACTGGATTCC | (SEQ ID NO: 31) |
| Sense strand | |
| 5′-GATCCCCGCACATGGAGGACTGGATTTTCAA | (SEQ ID NO: 32) |
| GAGAAATCCAGTCCTCCATGTGCTTTTT-3′ | |
| Antisense strand: | |
| 5′-AGCTAAAAAGCACATGGAGGACTGGATTTCT | (SEQ ID NO: 33) |
| CTTGAAAATCCAGTCCTCCATGTGCGGG-3′ | |

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. In one embodiment, the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program on the internet which can be found by entering http://, followed by biotools.idtdna.com/antisense/AntiSense.aspx, which will provide appropriate antisense sequences for nucleic acid sequences up to 10,000 nucleotides in length. Using this program with the sEH gene provides the following exemplar sequences:

| | | |
|---|---|---|
| 1) | UGUCCAGUGCCCACAGUCCU | (SEQ ID NO: 34) |
| 2) | UUCCCACCUGACACGACUCU | (SEQ ID NO: 35) |
| 3) | GUUCAGCCUCAGCCACUCCU | (SEQ ID NO: 36) |
| 4) | AGUCCUCCCGCUUCACAGA | (SEQ ID NO: 37) |
| 5) | GCCCACUUCCAGUUCCUUUCC | (SEQ ID NO: 38) |

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a sEH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996). Another subsequence, the h (hydrophobic) domain of signal peptides, was found to have similar cell membrane translocation characteristics (see, e.g., Lin et al., J. Biol. Chem. 270:14255-14258 (1995)). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

More recently, it has been discovered that siRNAs can be introduced into mammals without eliciting an immune response by encapsulating them in nanoparticles of cyclodextrin. Information on this method can be found by entering “www.” followed by “nature.com/news/2005/050418/full/050418-6.html.”

In another method, the nucleic acid is introduced directly into superficial layers of the skin or into muscle cells by a jet of compressed gas or the like. Methods for administering naked polynucleotides are well known and are taught, for example, in U.S. Pat. No. 5,830,877 and International Publication Nos. WO 99/52483 and 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus.

miRNAs and siRNAs differ in several ways: miRNA derive from points in the genome different from previously recognized genes, while siRNAs derive from mRNA, viruses or transposons, miRNA derives from hairpin structures, while siRNA derives from longer duplexed RNA, miRNA is conserved among related organisms, while siRNA usually is not, and miRNA silences loci other than that from which it derives, while siRNA silences the loci from which it arises. Interestingly, miRNAs tend not to exhibit perfect complementarity to the mRNA whose expression they inhibit. See, McManus et al., supra. See also, Cheng et al., Nucleic Acids Res. 33(4): 1290-7 (2005); Robins and Padgett, Proc Natl Acad Sci USA. 102(11):4006-9 (2005); Brennecke et al., PLoS Biol. 3(3):e85 (2005). Methods of designing miRNAs are known. See, e.g., Zeng et al., Methods Enzymol. 392:371-80 (2005); Krol et al., J Biol. Chem. 279(40):42230-9 (2004); Ying and Lin, Biochem Biophys Res Commun. 326(3):515-20 (2005).

### Therapeutic Administration

EETs and inhibitors of sEH can be prepared and administered in a wide variety of formulations for administration to the eyes. The formulations can be introduced onto or into the eye by, for example, applying the formulation to the eyelids or to the conjunctival sac in aqueous or viscous solutions or suspensions, in ointments, in small pellets, as fine powders, on cotton pledgets, by drug-impregnated contact lenses, by injection, by mechanical pumps, or by membrane release systems. In preferred forms, compounds for topical use in the methods of the present invention can be administered as eye drops, ointments, or small pellets to be placed under the eyelids. Accordingly, the methods of the invention permit administration of pharmaceutical compositions comprising a pharmaceutically acceptable carrier or excipient and a selected EET or sEH inhibitor, or combination thereof.

Administration of pharmacologically active agents to the eyes is well known, and considerable information is set forth in standard works, such as Zimmerman et al. (eds.), TEXTBOOK OF OCULAR PHARMACOLOGY, Lippincott Williams & Wilkins (1997); Jannus et al., (eds.), CLINICAL OCULAR PHARMACOLOGY, Butterworth-Heinemann (4th Ed., 2001), and Mauger and Craig, HAVENER'S OCULAR PHARMACOLOGY, Mosby-Year Book (6th Ed., 1994), Grosvenor, PRIMARY CARE OPTOMETRY, Butterworth-Heinemann, (4th Ed., 2001), Duvall and Kerschner, OPHTHALMIC MEDICATIONS AND PHARMACOLOGY, SLACK Inc., Thorofare, N.J. (1998), and Fechner and Teichmann, OCULAR THERAPEUTICS: PHARMACOLOGY AND CLINICAL APPLICATION, SLACK Inc., Thorofare, N.J. (1997). These well known techniques can be readily applied to prepare and administer EETs, sEHI, or combinations of EETs and one or more sEHIs to persons in need thereof.

For preparing pharmaceutical compositions from sEH inhibitors, or EETs, or both, pharmaceutically acceptable carriers can be either solid or liquid. The carriers may also act, for example, as diluents, binders, or preservatives.

Liquid form preparations include solutions, suspensions, and emulsions, for example, water or water/propylene glycol solutions. Other typical forms for administration of the EETs, sEHI, or combinations thereof are liquid paraffin, polyvinyl alcohol, povidine, carbomers, hypromellose, hydroxyethylcellulose, hydroxypropylcellulose, and carboxymethylcellulose.

Formulations for intravitreous injection are also known in the art. Intravitreal injection is typically performed in the outpatient setting using topical anesthesia and a small-bore needle (e.g., 27 or 30 gauge) to deliver the medication into the vitreous cavity of the eye via the pars plana portion of the globe. Typically, the EETs or sEHI, or combination are administered as a sterile, preservative-free aqueous solution, which may optionally contain sodium chloride, monobasic sodium phosphate monohydrate, dibasic sodium phosphate heptahydrate, hydrochloric acid, and/or sodium hydroxide and other agents to adjust the viscosity and pH.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as vials or ampoules.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the sEH inhibitor, or EET, or both, is employed in reducing intraocular pressure, alleviating dry eye syndrome, or for slowing or reversing the progression of AMD, and especially wet AMD. The dosage of the specific compound for treatment depends on many factors that are well known to those skilled in the art. They include for example, the route of administration and the potency of the particular compound. As noted in a preceding section, an anti-VEGF siRNA injected into the eye has proven effective in a Phase I clinical trial on wet AMD. Thus, administration of siRNA directed to sEH is expected to be useful in treating the eye conditions discussed herein.

In some aspects of the invention, the sEH inhibitor, EET, or combination thereof, is dissolved or suspended in a suitable solvent, such as water, ethanol, or saline, and administered as an aerosol of fine particles by breaking a fluid into fine droplets and dispersing them into a flowing stream of gas. Typically, such aerosols develop approximately 15 to 30 microliters of aerosol per liter of gas in finely divided droplets with volume or mass median diameters in the range of 2 to 4 micrometers. Predominantly, water or saline solutions are used with low solute concentrations, typically ranging from 1.0 to 5.0 mg/mL.

As noted, drugs may be applied to the eyelids or instilled in the conjunctival sac in aqueous or viscous solutions or suspensions, in ointments, as fine powders, on cotton pledgets, by drug-impregnated contact lenses, by injection, by mechanical pumps, or by membrane release systems. In contrast to systemic administration, the ocular concentration after topical administration is high. Dilution of the drug by tears, overflow onto the cheek, and excretion through the nasolacrimal system limit tissue concentration. Placing the drug beneath a contact lens, applying a cotton pledget, or applying a collagen shield saturated with the drug to the eye prolongs the contact and aids penetration.

Without further elaboration, it is believed that one skilled in the art can, using the preceding description, practice the present invention to its fullest extent.

### EXAMPLES

### Example 1

This Example sets forth materials and methods used in studies underlying the present invention.

All animal care and procedures were approved by the University of California, Davis Institutional Animal Care and Use Committee and complied with the Guide for the Care and Use of Laboratory Animals (NIH publication No. 80-213, 1985).

Female New Zealand white rabbits (2.7-3.0 kg) were obtained from Harlan (Indianapolis, Ind.). On test day, rabbits were lightly tranquilized with torbugesic/acepromazine at 0.04 mg/kg, administered subcutaneously. Ten to twenty minutes after administration of the tranquilizer, a drop of proparacaine hydrochloride (0.5% ophthalmic solution) was instilled in each eye to numb the eye prior to taking the intraocular pressure measurement. After 2-5 min, intraocular pressure in each eye (right and left) was measured using a tonometer (Tono-Pen®, Mentor Opthalmics, Santa Barbara, Calif.). Measurements were taken every 3-5 minutes to obtain baseline pressure. Then various treatments were applied (right eye) and after 3-5 minutes measurements were again taken every 3-5 minutes. Data are expressed as the change in intraocular pressure (“IOP”) in the treated eye compared to the control eye (IOP in treated eye—IOP in control eye). The first treatments were compounds dissolved in a neutral artificial tear solution containing 1% dimethyl sulfoxide to enhance solubility. DMSO is also known to aid penetration through the skin. For a treatment to be effective, it must penetrate the cornea. Later treatments utilized subconjunctival injection. This delivers the compound below the conjunctiva where it is rapidly absorbed, avoiding issues of absorption.

Treatments: Each rabbit was administered one of the following treatments: (1). Epoxyeicosatrienoic acids (EETs) only, 100 μg/mL carboxylmethyl cellulose artificial tears containing 1% dimethyl sulfoxide (DMSO), 50 μL/eye (2). AUDA only, 100 μg/mL carboxylmethyl cellulose artificial tears containing 1% DMSO, 50 μL/eye (3). EETs only, 10 μg/mL carboxylmethyl cellulose artificial tears containing 1% DMSO, 0.1 cc/eye administered subconjunctivally (4). AUDA only, 10 μg/mL carboxylmethyl cellulose artificial tears containing 1% DMSO, 0.1 cc/eye administered subconjunctivally (5) EETs only, 100 μg/mL carboxylmethyl cellulose artificial tears containing 1% DMSO, 0.1 cc/eye administered subconjunctivally (6) AUDA only, 100 μg/mL carboxylmethyl cellulose artificial tears containing 1% DMSO, 0.1 cc/eye administered subconjunctivally (7) vehicle control administered subconjunctivally (8) Lumigan® (brimatoprost 0.03% ophthalmic solution, Allergan, Inc., Irvine, Calif.) instilled in 2 drops spaced 10 min apart.

Compounds used for the topical treatment of eye diseases become clinically useful only if they can penetrate the intact epithelium of the cornea and conjunctiva and achieve clinically useful concentrations within the eye. Thus for these trials we used both instillation by drops and by subconjunctival injections.

### Example 2

This Example sets forth the results of studies underlying the present invention.

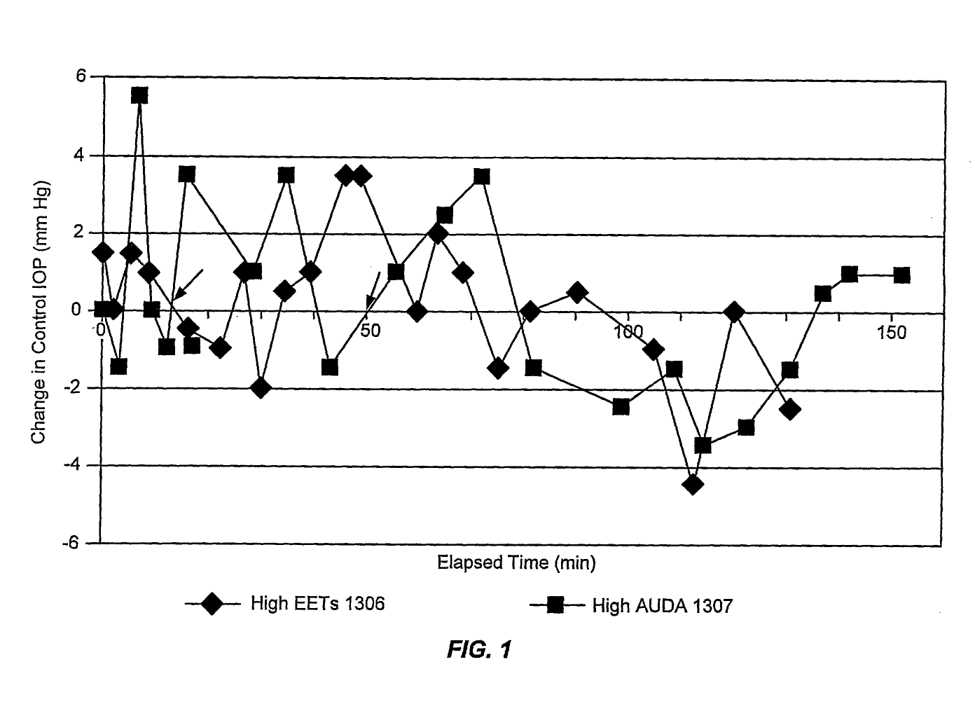

For Rabbit 1306, IOP measurements were taken several times over 12 minutes (FIG. 1). At 12 minutes, the high EETs solution was instilled in the eye topically (arrow). IOP measurements were taken for a total of about 130 min. Following initial instillation, IOP fluctuated, but showed a definite declining trend after 60 min, about 45 minutes after treatment.

For Rabbit 1307, IOP measurements were taken several times over 50 minutes. At 50 minutes, the high dose AUDA solution was instilled in the eye topically (arrow). IOP measurements were taken for a total of about 155 min. Following initial instillation, the IOP fluctuated, but showed a distinct decline in pressure after about 70 minutes, about 20 minutes after treatment.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQ ID NO.: 1 Amino acid sequence of human soluble
epoxide
MTLRGAVFDLDGVLALPAVFGVLGRTEEALALPRGLLNDAFQKGGPEGAT
TRLMKGEITLSQWIPLMEENCRKCSETAKVCLPKNFSIKEIFDKAISARK
INRPMLQAALMLRKKGFTTAILTNTWLDDRAERDGLAQLMCELKMHFDFL
IESCQVGMVKPEPQIYKFLLDTLKASPSEVVFLDDIGANLKPARDLGMVT
ILVQDTDTALKELEKVTGIQLLNTPAPLPTSCNPSDMSHGYVTVKPRVRL
HFVELGWPAVCLCHGFPESWYSWRYQIPALAQAGYRVLAMDMKGYGESSA
PPEIEEYCMEVLCKEMVTFLDKLGLSQAVFIGHDWGGMLVWYMALFYPER
VRAVASLNTPFIPANPNMSPLESIKANPVFDYQLYFQEPGVAEAELEQNL
SRTFKSLFRASDESVLSMHKVCEAGGLFVNSPEEPSLSRMVTEEEIQFYV
QQFKKSGFRGPLNWYRNMERNWKWACKSLGRKILIPALMVTAEKDFVLVP
QMSQHMEDWIPHLKRGHIEDCGHWTQMDKPTEVNQILIKWLDSDARNPPV
VSKM
SEQ ID NO.: 2 Nucleic acid sequence encoding human
soluble epoxide
atgacgctg cgcggcgccg tcttcgacct tgacggggtg
ctggcgctgc cagcggtgtt cggcgtcctc ggccgcacgg
aggaggccct ggcgctgccc agaggacttc tgaatgatgc
tttccagaaa gggggaccag agggtgccac tacccggctt
atgaaaggag agatcacact ttcccagtgg ataccactca
tggaagaaaa ctgcaggaag tgctccgaga ccgctaaagt
ctgcctcccc aagaatttct ccataaaaga aatctttgac
aaggcgattt cagccagaaa gatcaaccgc cccatgctcc
aggcagctct catgctcagg aagaaaggat tcactactgc
catcctcacc aacacctggc tggacgaccg tgctgagaga
gatggcctgg cccagctgat gtgtgagctg aagatgcact
ttgacttcct gatagagtcg tgtcaggtgg gaatggtcaa
acctgaacct cagatctaca agtttctgct ggacaccctg
aaggccagcc ccagtgaggt cgtttttttg gatgacatcg
gggctaatct gaagccagcc cgtgacttgg gaatggtcac
catcctggtc caggacactg acacggccct gaaagaactg
gagaaagtga ccggaatcca gcttctcaat accccggccc
ctctgccgac ctcttgcaat ccaagtgaca tgagccatgg
gtacgtgaca gtaaagccca gggtccgtct gcattttgtg
gagctgggct ggcctgctgt gtgcctctgc catggatttc
ccgagagttg gtattcttgg aggtaccaga tccctgctct
ggcccaggca ggttaccggg tcctagctat ggacatgaaa
ggctatggag agtcatctgc tcctcccgaa atagaagaat
attgcatgga agtgttatgt aaggagatgg taaccttcct
ggataaactg ggcctctctc aagcagtgtt cattggccat
gactggggtg gcatgctggt gtggtacatg gctctcttct
accccgagag agtgagggcg gtggccagtt tgaatactcc
cttcatacca gcaaatccca acatgtcccc tttggagagt
atcaaagcca acccagtatt tgattaccag ctctacttcc
aagaaccagg agtggctgag gctgaactgg aacagaacct
gagtcggact ttcaaaagcc tcttcagagc aagcgatgag
agtgttttat ccatgcataa agtctgtgaa gcgggaggac
tttttgtaaa tagcccagaa gagcccagcc tcagcaggat
ggtcactgag gaggaaatcc agttctatgt gcagcagttc
aagaagtctg gtttcagagg tcctctaaac tggtaccgaa
acatggaaag gaactggaag tgggcttgca aaagcttggg
acggaagatc ctgattccgg ccctgatggt cacggcggag
aaggacttcg tgctcgttcc tcagatgtcc cagcacatgg
aggactggat tccccacctg aaaaggggac acattgagga
ctgtgggcac tggacacaga tggacaagcc aaccgaggtg
aatcagatcc tcattaagtg gctggattct gatgcccgga
acccaccggt ggtctcaaag atgtag

What is claimed is:

1. A method of reducing intraocular pressure in an eye of an individual in need thereof, said method comprising administering to said individual by administering to said eye an effective amount of an agent or agents selected from the group consisting of a cis-epoxyeicosatrienoic acid (“EET”), an inhibitor of soluble epoxide hydrolase (“sEH”), and a combination of an EET and an inhibitor of sEH, thereby reducing intraocular pressure.

2. A method of claim 1, wherein the administration is by topical application of a liquid comprising said agent or agents.

3. A method of claim 2, wherein the topical application is by instilling of the liquid into the conjunctival sac of the eye.

4. A method of claim 1, wherein the administration is by topical administration of an ointment comprising said agent or agents.

5. A method of claim 1, wherein the administration is by injection into the eye.

6. A method of claim 1, wherein the individual has glaucoma.

7. A method of claim 1, wherein said inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea, and an alkylpiperidine.

8. A method of claim 1, wherein said EET is selected from the group consisting of 14,15-EET, 8,9-EET and 11,12-EET.

9. A method of claim 1, wherein the EET or the inhibitor of sEH, or both, are in a material which releases the EET, or inhibitor, or both, over time.

10. A method of claim 1, wherein the inhibitor of sEH is a small interfering RNA which inhibits expression of sEH.

11. A method of alleviating “dry eye syndrome” in an individual in need thereof, said method comprising administration to an eye of said individual an effective amount of an agent or agents selected from the group consisting of a cis-epoxyeicosatrienoic acid (“EET”), an inhibitor of soluble epoxide hydrolase (“sEH”), and a combination of an EET and an inhibitor of sEH, thereby alleviating said dry eye syndrome.

12. A method of claim 11, wherein the administration is by topical application of a liquid comprising said agent or agents.

13. A method of claim 12, wherein the topical application is by instilling of the liquid into the conjunctival sac of the eye.

14. A method of claim 11, wherein the administration is by topical administration of an ointment comprising said agent or agents.

15. A method of claim 11, wherein the administration is by topical administration of a small pellet comprising said agent or agents.

16. A method of claim 11, wherein the administration is by release from a punctal plug inserted into a tear duct.

17. A method of claim 11, wherein the administration is by injection into the eye.

18. A method of claim 11, wherein said inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea and an alkylpiperidine.

19. A method of claim 11, wherein said EET is selected from the group consisting of 14,15-EET, 8,9-EET and 11,12-EET.

20. A method of claim 11, wherein the EET or the inhibitor of sEH, or both, are in a material which releases the EET, or inhibitor, or both, over time.

21. A method of claim 11, wherein the inhibitor of sEH is a small interfering RNA which inhibits expression of sEH.

22. A method of reducing progression of a condition selected from the group consisting of (a) age-related macular degeneration (“AMD”), (b) diabetic retinopathy, and (c) rejection of a corneal graft, in an eye of an individual in need thereof, said method comprising administering to said individual by administration to said eye an effective amount of an agent or agents selected from the group consisting of a cis-epoxyeicosatrienoic acid (“EET”), an inhibitor of soluble epoxide hydrolase (“sEH”), and a combination of an EET and an inhibitor of sEH, thereby reducing progression of AMD, diabetic retinopathy, or rejection of a corneal graft in said eye.

23. A method of claim 22, wherein the administration is by topical application of a liquid comprising said agent or agents.

24. A method of claim 22, wherein the topical application is by instilling of the liquid into the conjunctival sac of the eye.

25. A method of claim 22, wherein the administration is by topical administration of an ointment comprising said agent or agents.

26. A method of claim 22, wherein the administration is by injection into the eye.

27. A method of claim 22, wherein the inhibitor of sEH is a small interfering RNA which inhibits expression of sEH.

28. A method of claim 22, wherein said inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea and an alkylpiperidine.

29. A method of claim 22, wherein said EET is selected from the group consisting of 14,15-EET, 8,9-EET and 11,12-EET.

30. A method of claim 22, wherein the EET or the inhibitor of sEH, or both, are in a material which releases the EET, or inhibitor, or both, over time.

31. A method of claim 22, wherein the condition is AMD.

32. A method of claim 22, wherein the condition is diabetic retinopathy.

33. A method of claim 22, wherein the condition is rejection of a corneal graft.

34. A method of claim 22, wherein said individual in need thereof does not have an inflammatory disorder other than AMD or has been treated for an inflammatory disorder other than AMD with an agent which is not an EET or an inhibitor of sEH.

35. A method of reducing intraocular pressure, alleviating dry eye syndrome, reducing progression of age-related macular degeneration (“AMD”), reducing diabetic retinopathy, or reducing rejection of a corneal graft in an eye of an individual in need thereof, said method comprising systemic administration to said individual of an effective amount of an agent or agents selected from the group consisting of an inhibitor of soluble epoxide hydrolase (“sEH”), and a combination of a cis-epoxyeicosatrienoic acid (“EET”) and an inhibitor of sEH, thereby reducing intraocular pressure, alleviating dry eye syndrome, reducing progression of AMD, reducing diabetic retinopathy, or reducing rejection of a corneal graft.

36. A method of claim 35, wherein said inhibitor of sEH is selected from the group consisting of an adamantyl dodecyl urea, 12-(3-adamantan-1-yl-ureido)dodecanoic acid, 12-(3-adamantan-1-yl-ureido)dodecanoic acid, and adamantan-1-yl-3-{5-[2-(2-ethoxyethoxy)ethoxy]pentyl}urea, and an alkylpiperidine.

37. A method of claim 35, wherein said EET is selected from the group consisting of 14,15-EET, 8,9-EET and 11,12-EET.

38. A method of claim 35, wherein the EET or the inhibitor of sEH, or both, are in a material which releases the EET, or inhibitor, or both, over time.

39. A method of claim 35, wherein the individual has glaucoma.

40. A method of claim 35, wherein said individual has dry eye syndrome.

41. A method of claim 35, wherein said individual has AMD.

42. A method of claim 35, wherein said AMD is wet AMD.

43. A method of claim 35, wherein said inhibitor of sEH is an isolated nucleic acid which inhibits expression of a gene encoding soluble epoxide hydrolase (“sEH”).


SEQUENCE LISTING (38 sequences)

SEQ ID NO: 1 - protein, 554 aa, Homo sapiens
  human soluble epoxide hydrolase (sEH, cytosolic EH)
MTLRGAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGWPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 2 - DNA, 1665 bp, Homo sapiens
  human soluble epoxide hydrolase (sEH, cytosolic EH)
atgacgctgcgcggcgccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctggcctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtag

SEQ ID NO: 3 - DNA, 23 bp, Artificial Sequence
  Description of Artificial Sequencehuman soluble epoxide hydrolase (sEH, cytosolic EH) small interfering RNA (siRNA) target sequence
cagtgttcattggccatgactgg

SEQ ID NO: 4 - DNA, 21 bp, Artificial Sequence
  Description of Combined DNA/RNA Moleculehuman soluble epoxide hydrolase (sEH, cytosolic EH) sense small interfering RNA (siRNA)
guguucauuggccaugacutt

SEQ ID NO: 5 - DNA, 21 bp, Artificial Sequence
  Description of Combined DNA/RNA Moleculehuman soluble epoxide hydrolase (sEH, cytosolic EH) antisense small interfering RNA (siRNA)
agucauggccaaugaacactt

SEQ ID NO: 6 - DNA, 23 bp, Artificial Sequence
  Description of Artificial Sequencehuman soluble epoxide hydrolase (sEH, cytosolic EH) small interfering RNA (siRNA) target sequence
gaaaggctatggagagtcatctg

SEQ ID NO: 7 - DNA, 21 bp, Artificial Sequence
  Description of Combined DNA/RNA Moleculehuman soluble epoxide hydrolase (sEH, cytosolic EH) sense small interfering RNA (siRNA)
aaggcuauggagagucauctt

SEQ ID NO: 8 - DNA, 21 bp, Artificial Sequence
  Description of Combined DNA/RNA Moleculehuman soluble epoxide hydrolase (sEH, cytosolic EH) antisense small interfering RNA (siRNA)
gaugacucuccauagccuutt

SEQ ID NO: 9 - DNA, 23 bp, Artificial Sequence
  Description of Artificial Sequencehuman soluble epoxide hydrolase (sEH, cytosolic EH) small interfering RNA (siRNA) target sequence
aaaggctatggagagtcatctgc

SEQ ID NO: 10 - DNA, 21 bp, Artificial Sequence
  Description of Combined DNA/RNA Moleculehuman soluble epoxide hydrolase (sEH, cytosolic EH) sense small interfering RNA (siRNA)
aggcuauggagagucaucutt

SEQ ID NO: 11 - DNA, 21 bp, Artificial Sequence
  Description of Combined DNA/RNA Moleculehuman soluble epoxide hydrolase (sEH, cytosolic EH) antisense small interfering RNA (siRNA)
agaugacucuccauagccutt

SEQ ID NO: 12 - DNA, 23 bp, Artificial Sequence
  Description of Artificial Sequencehuman soluble epoxide hydrolase (sEH, cytosolic EH) small interfering RNA (siRNA) target sequence
caagcagtgttcattggccatga

SEQ ID NO: 13 - DNA, 21 bp, Artificial Sequence
  Description of Combined DNA/RNA Moleculehuman soluble epoxide hydrolase (sEH, cytosolic EH) sense small interfering RNA (siRNA)
agcaguguucauuggccautt

SEQ ID NO: 14 - DNA, 21 bp, Artificial Sequence
  Description of Combined DNA/RNA Moleculehuman soluble epoxide hydrolase (sEH, cytosolic EH) antisense small interfering RNA (siRNA)
auggccaaugaacacugcutt

SEQ ID NO: 15 - DNA, 23 bp, Artificial Sequence
  Description of Artificial Sequencehuman soluble epoxide hydrolase (sEH, cytosolic EH) small interfering RNA (siRNA) target sequence
cagcacatggaggactggattcc

SEQ ID NO: 16 - DNA, 21 bp, Artificial Sequence
  Description of Combined DNA/RNA Moleculehuman soluble epoxide hydrolase (sEH, cytosolic EH) sense small interfering RNA (siRNA)
gcacauggaggacuggauutt

SEQ ID NO: 17 - DNA, 21 bp, Artificial Sequence
  Description of Combined DNA/RNA Moleculehuman soluble epoxide hydrolase (sEH, cytosolic EH) antisense small interfering RNA (siRNA)
aauccaguccuccaugugctt

SEQ ID NO: 18 - DNA, 9 bp, Artificial Sequence
  Description of Artificial Sequencehairpin RNA linker section, short spacer
ttcaagaga

SEQ ID NO: 19 - DNA, 23 bp, Artificial Sequence
  Description of Artificial Sequencehuman soluble epoxide hydrolase (sEH, cytosolic EH) short hairpin small interfering RNA (siRNA) target sequence
cagtgttcattggccatgactgg

SEQ ID NO: 20 - DNA, 59 bp, Artificial Sequence
  Description of Artificial Sequencehuman soluble epoxide hydrolase (sEH, cytosolic EH) short hairpin small interfering RNA (siRNA) sense strand
gatccccgtgttcattggccatgactttcaagagaagtcatggccaatgaacacttttt

SEQ ID NO: 21 - DNA, 59 bp, Artificial Sequence
  Description of Artificial Sequencehuman soluble epoxide hydrolase (sEH, cytosolic EH) short hairpin small interfering RNA (siRNA) antisense strand
agctaaaaagtgttcattggccatgacttctcttgaaagtcatggccaatgaacacggg

SEQ ID NO: 22 - DNA, 23 bp, Artificial Sequence
  Description of Artificial Sequencehuman soluble epoxide hydrolase (sEH, cytosolic EH) short hairpin small interfering RNA (siRNA) target sequence
gaaaggctatggagagtcatctg

SEQ ID NO: 23 - DNA, 59 bp, Artificial Sequence
  Description of Artificial Sequencehuman soluble epoxide hydrolase (sEH, cytosolic EH) short hairpin small interfering RNA (siRNA) sense strand
gatccccaaggctatggagagtcatcttcaagagagatgactctccatagccttttttt

SEQ ID NO: 24 - DNA, 59 bp, Artificial Sequence
  Description of Artificial Sequencehuman soluble epoxide hydrolase (sEH, cytosolic EH) short hairpin small interfering RNA (siRNA) antisense strand
agctaaaaaaaggctatggagagtcatctctcttgaagatgactctccatagccttggg

SEQ ID NO: 25 - DNA, 23 bp, Artificial Sequence
  Description of Artificial Sequencehuman soluble epoxide hydrolase (sEH, cytosolic EH) short hairpin small interfering RNA (siRNA) target sequence
aaaggctatggagagtcatctgc

SEQ ID NO: 26 - DNA, 59 bp, Artificial Sequence
  Description of Artificial Sequencehuman soluble epoxide hydrolase (sEH, cytosolic EH) short hairpin small interfering RNA (siRNA) sense strand
gatccccaggctatggagagtcatctttcaagagaagatgactctccatagcctttttt

SEQ ID NO: 27 - DNA, 59 bp, Artificial Sequence
  Description of Artificial Sequencehuman soluble epoxide hydrolase (sEH, cytosolic EH) short hairpin small interfering RNA (siRNA) antisense strand
agctaaaaaaggctatggagagtcatcatctcttgaaagatgactctccatagcctggg

SEQ ID NO: 28 - DNA, 23 bp, Artificial Sequence
  Description of Artificial Sequencehuman soluble epoxide hydrolase (sEH, cytosolic EH) short hairpin small interfering RNA (siRNA) target sequence
caagcagtgttcattggccatga

SEQ ID NO: 29 - DNA, 59 bp, Artificial Sequence
  Description of Artificial Sequencehuman soluble epoxide hydrolase (sEH, cytosolic EH) short hairpin small interfering RNA (siRNA) sense strand
gatccccagcagtgttcattggccatttcaagagaatggccaatgaacactgctttttt

SEQ ID NO: 30 - DNA, 59 bp, Artificial Sequence
  Description of Artificial Sequencehuman soluble epoxide hydrolase (sEH, cytosolic EH) short hairpin small interfering RNA (siRNA) antisense strand
agctaaaaaagcagtgttcattggccattctcttgaaatggccaatgaacactgctggg

SEQ ID NO: 31 - DNA, 23 bp, Artificial Sequence
  Description of Artificial Sequencehuman soluble epoxide hydrolase (sEH, cytosolic EH) short hairpin small interfering RNA (siRNA) target sequence
cagcacatggaggactggattcc

SEQ ID NO: 32 - DNA, 59 bp, Artificial Sequence
  Description of Artificial Sequencehuman soluble epoxide hydrolase (sEH, cytosolic EH) short hairpin small interfering RNA (siRNA) sense strand
gatccccgcacatggaggactggattttcaagagaaatccagtcctccatgtgcttttt

SEQ ID NO: 33 - DNA, 59 bp, Artificial Sequence
  Description of Artificial Sequencehuman soluble epoxide hydrolase (sEH, cytosolic EH) short hairpin small interfering RNA (siRNA) antisense strand
agctaaaaagcacatggaggactggatttctcttgaaaatccagtcctccatgtgcggg

SEQ ID NO: 34 - RNA, 20 nt, Artificial Sequence
  Description of Artificial Sequencehuman soluble epoxide hydrolase (sEH, cytosolic EH) antisense sequence
uguccagugcccacaguccu

SEQ ID NO: 35 - RNA, 20 nt, Artificial Sequence
  Description of Artificial Sequencehuman soluble epoxide hydrolase (sEH, cytosolic EH) antisense sequence
uucccaccugacacgacucu

SEQ ID NO: 36 - RNA, 20 nt, Artificial Sequence
  Description of Artificial Sequencehuman soluble epoxide hydrolase (sEH, cytosolic EH) antisense sequence
guucagccucagccacuccu

SEQ ID NO: 37 - RNA, 19 nt, Artificial Sequence
  Description of Artificial Sequencehuman soluble epoxide hydrolase (sEH, cytosolic EH) antisense sequence
aguccucccgcuucacaga

SEQ ID NO: 38 - RNA, 21 nt, Artificial Sequence
  Description of Artificial Sequencehuman soluble epoxide hydrolase (sEH, cytosolic EH) antisense sequence
gcccacuuccaguuccuuucc